(19) United States
(12) Patent Application Publication
(10) Pub. No.: US 2024/0293293 A1
(43) Pub. Date: Sep. 5, 2024

(54) RE-USABLE AND AFFIXABLE FACE COVERING OR MASK

(71) Applicant: Shawsheen Rubber Co., Inc., Andover, MA (US)

(72) Inventors: Gary Jay Litman, Frarningham, MA (US); Nathan William Smith, Luneburg, MA (US)

(21) Appl. No.: 18/593,164

(22) Filed: Mar. 1, 2024

Related U.S. Application Data
(60) Provisional application No. 63/449,649, filed on Mar. 3, 2023.

(51) Int. Cl. A61K 8/02; A61K 8/67; A61K 8/73; A61Q 19/08

(52) U.S. Cl. CPC A61K 8/0212; A61K 8/678; A61K 8/735; A61Q 19/08; A61K 2121/00

(57) ABSTRACT

A therapeutic skin mask having: an elastomeric film barrier layer, and a pressure sensitive adhesive (PSA) disposed on a first side of the elastomeric film barrier layer, the PSA having a therapeutic ingredient configured to treat an area of skin upon which the PSA is applied; wherein the elastomeric film barrier layer includes a shape configured to cover the area of skin.

### CROSS REFERENCE TO RELATED APPLICATION

This application claims the benefit of an earlier filing date from U.S. Provisional Application Ser. No. 63/449,649 filed Mar. 3, 2023, the entire disclosure of which is incorporated herein by reference.

### BACKGROUND

Facial skin is typically exposed to environment challenges such as sun rays, wind, humidity variations, and temperature extremes. These challenges can cause various insults to the facial skin such a wrinkles and “crow's feet.” In addition, aging can also cause insults to the facial skin such as aging blemishes. Hence, people affected by these insults would appreciate therapies to treat insults to the facial skin. In particular, the therapies would include application of therapeutic substances to the facial skin.

### SUMMARY

Disclosed is a therapeutic skin mask including: an elastomeric film barrier layer; and a pressure sensitive adhesive (PSA) disposed on a first side of the elastomeric film barrier layer, the PSA including a therapeutic ingredient configured to treat an area of skin upon which the PSA is applied; wherein the elastomeric film barrier layer includes a shape configured to cover the area of skin.

In addition to one or more aspects of the mask or as an alternate, the elastomeric film barrier layer includes a blend of polyurethane and polyester.

In addition to one or more aspects of the mask or as an alternate, the PSA includes a 100% solid, solvent-free silicone gel.

In addition to one or more aspects of the mask or as an alternate, the therapeutic ingredient includes at least one of high molecular weight hyaluronic acid, low molecular weight hyaluronic acid, collagen, EGCG, CoQ10, argireline, bakuchiol, or vitamin E.

In addition to one or more aspects of the mask or as an alternate, the elastomeric film barrier layer is not directly or indirectly coupled to a substrate on a second side of the elastomeric film barrier layer opposing the first side.

In addition to one or more aspects of the mask or as an alternate, the mask includes a fabric substrate disposed on a second side on the elastomeric film barrier layer opposite the first side.

In addition to one or more aspects of the mask or as an alternate, the fabric substrate is stretchable in multiple directions.

In addition to one or more aspects of the mask or as an alternate, the mask includes a release liner configured to be applied to the PSA side of the therapeutic skin mask in response to the therapeutic skin mask not being used.

In addition to one or more aspects of the mask or as an alternate, the release liner includes a PET film coated on one side with a fluorosilicone release coating.

In addition to one or more aspects of the mask or as an alternate, the shape of the elastomeric film barrier layer is such that the elastomeric film barrier layer is configured to cover at least one of a face, a partial area of the face, a neck, a hand, a knee, or a décolletage area.

In addition to one or more aspects of the mask or as an alternate, the elastomeric film barrier layer is configured to cover the face and defines one or more openings for eyes, nostrils, or a mouth.

In addition to one or more aspects of the mask or as an alternate, the shape includes contours conforming to an area under an eye.

Further disclosed is a method for a therapeutic treatment an area of skin, the method including: obtaining a therapeutic skin mask, the therapeutic skin mask including an elastomeric film barrier layer and a pressure sensitive adhesive (PSA) disposed on a first side of the elastomeric film barrier layer, the PSA including a therapeutic ingredient configured to treat a skin area upon which the PSA is applied, wherein the elastomeric film barrier layer includes a shape configured to cover the area of skin; and applying the therapeutic skin mask to the area of skin.

In addition to one or more aspects of the method or as an alternate, the method includes: removing the therapeutic skin mask from the area of skin; and placing the therapeutic skin mask with a side having the PSA on a release liner.

In addition to one or more aspects of the method or as an alternate, the method includes removing the therapeutic skin mask from the release liner and applying again the therapeutic skin mask to the area of skin.

In addition to one or more aspects of the method or as an alternate, the method includes repeating the removing the therapeutic skin mask from the area of skin, and the applying again the therapeutic skin mask to the area of skin, for a selected number of times or until at least one of the PSA loses adhesiveness or the therapeutic ingredient is depleted.

In addition to one or more aspects of the method or as an alternate, the elastomeric film barrier layer is not directly or indirectly coupled to a substrate on a second side of the elastomeric film barrier layer opposing the first side.

In addition to one or more aspects of the method or as an alternate, a fabric substrate disposed on a second side on the elastomeric film barrier layer opposite the first side.

### BRIEF DESCRIPTION OF THE DRAWINGS

The following descriptions should not be considered limiting in any way. With reference to the accompanying drawings, like elements are numbered alike:

FIG. 1 illustrates an outline of one embodiment of a facial therapeutic mask;

FIG. 2 illustrates an outline of another embodiment of the facial therapeutic mask;

FIG. 3 illustrates an outline of yet another embodiment of the facial therapeutic mask;

FIG. 4 illustrates a cross-sectional view of a first composite material of the facial therapeutic mask;

FIG. 5 illustrates a perspective view of the first composite material of the facial therapeutic mask;

FIG. 6 illustrates a cross-sectional view of a second composite material of the facial therapeutic mask;

FIG. 6 illustrates a cross-sectional view of a second composite material of the facial therapeutic mask;

FIG. 7 illustrates a perspective view of the second composite material of the facial therapeutic mask; and

FIG. 8 is a flow chart for a method for a therapeutic treatment of an area of skin.

### DETAILED DESCRIPTION

A detailed description of one or more embodiments of the disclosed apparatus and method are presented herein by way of exemplification and not limitation with reference to the Figures.

Disclosed is a transdermal delivery system for applying a therapeutic substance (inherently can include multiple substances) to facial skin, neck skin, and/or décolletage area skin, but which can also be applied to other locations of the body for the same desired effect in that area of skin. In particular, the transdermal delivery system includes a facial therapeutic covering or mask that is applied to and adherable to a face or other skin areas. Here, the term “facial mask” is intended to also be inclusive of a therapeutic covering for treatment of skin areas other than the face such as the neck and décolletage area. The therapeutic facial mask includes a pressure sensitive adhesive (PSA) that adheres the mask to the face when applied with little pressure (i.e., an amount of pressure that does not cause discomfort). The PSA is infused, embedded, or imbued with the therapeutic substance. Hence, the therapeutic substance is constantly applied to and diffused to the facial skin over time as long as the mask is adhered to the face such as overnight while sleeping. The facial therapeutic mask is re-usable. Accordingly, the transdermal delivery system includes a removeable liner that can be applied to the side of the mask having the PSA to store and protect the mask while not in use.

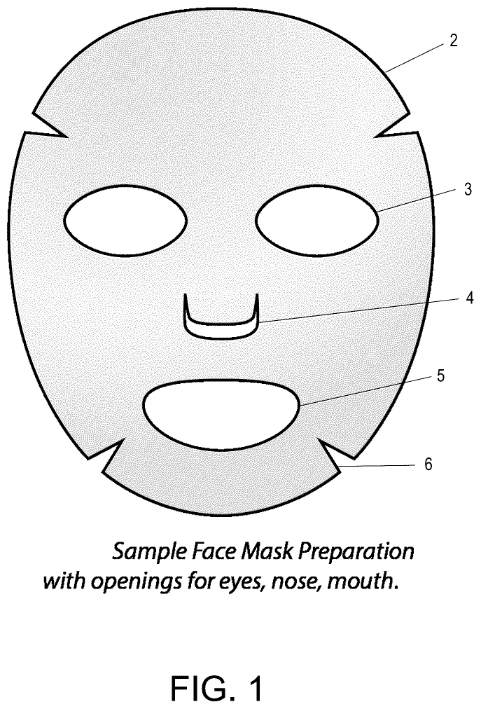

FIG. 1 illustrates an embodiment of a facial therapeutic mask 2 for being applied to a facial area. In the embodiment of FIG. 1, the facial therapeutic mask 2 defines an opening 3 for each of the eyes, an opening 4 for the nostrils, and an opening 5 for the mouth. The shape of the facial therapeutic mask 2 substantially follows the contours of the human face. Cutouts 6 or relief cuts along the perimeter of the mask 2 allow for shaping the mask 2 to allow for the curvature of the face to facilitate easy application. For covering the face, the mask 2 may extend vertically from the hairline to the chin and horizontally from ear to ear. Shorter ranges vertically and/or horizontally may also be used. Other shapes may also be used to cover other human skin areas of interest such as a square or rectangular shape for covering the face, forehead, neck hands, or décolletage areas. In addition, curved shapes that conform with contours of local areas of the face (such as below the eyes for example) or other skin areas may also be used. In general, curved shapes may conform with contours of local skin areas of interest.

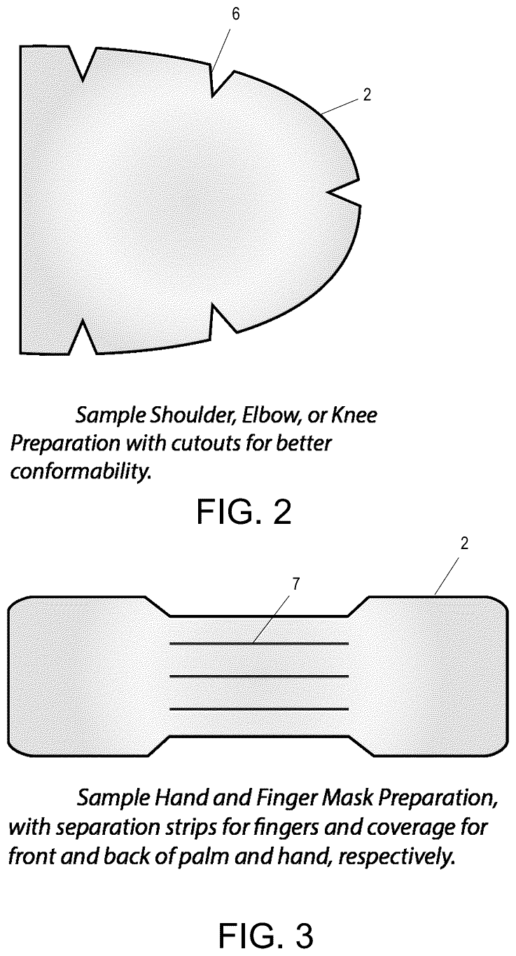

FIG. 2 illustrates an embodiment of the mask 2 for covering a shoulder, elbow, or knee with perimeter cutouts 6 allowing for shaping to conform to curvatures of the body.

FIG. 3 illustrates an embodiment of the mask 2 for covering a finger, a palm of a hand, or a back of a hand with separation strips 7 to allow for conforming to the curvature of a finger.

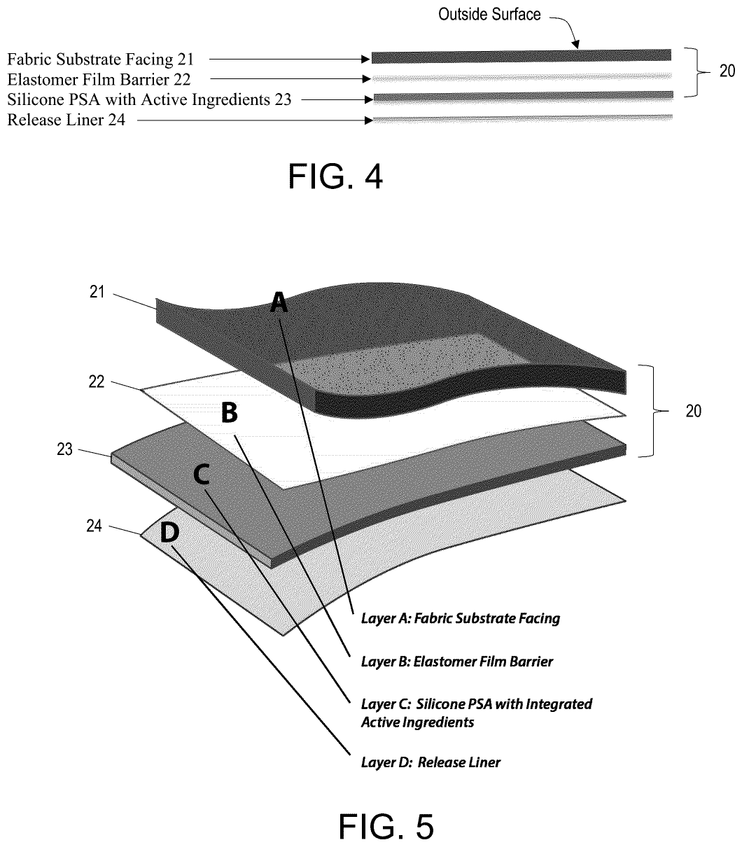

The facial therapeutic mask 2 is made of multiple layers of different materials or substances to form a sheet of a composite material 20. FIG. 4 illustrates a cross-sectional view of an embodiment of the composite material 20 of the facial therapeutic mask 2, while FIG. 5 provides a perspective view. For clarity, the layers are illustrated as being separated but are intended to be disposed one upon an adjacent other. The composite material 20 includes a substrate layer 21, an elastomer film barrier layer 22 disposed on a first side of the substrate layer 21, and a silicone pressure sensitive adhesive (PSA) with active therapeutic ingredients layer 23 disposed upon the elastomer film barrier layer 22. A release liner 24 is also illustrated for being disposed on the silicone pressure sensitive adhesive (PSA) with active therapeutic ingredients layer 23 for storage of the facial therapeutic mask 2. In one or more embodiments, the following material weights are used for the layers of the composite material 20: the fabric substrate layer 21—150 to 250 gsm (grams/square meter); the elastomer film barrier layer 22—25 to 45 gsm; the silicone PSA with active therapeutic ingredients layer 23—250 to 400 gsm; and release liner 24—100 to 125 gsm.

In one or more embodiments, the substrate layer 21 is an omnidirectional elastic or stretch substrate is used as a carrier for the PSA to allow for better conformability to the face and natural curvature of any anatomical location where the user applied the mask, and improved comfort while wearing for prolonged periods of time as opposed to a non-stretchable fabric. The omnidirectional elastic substrate can be made of an elastic knit fabric capable of stretch in all four primary directions (i.e., longitudinal and transverse stretch) such as Spandex fabric or an elastomeric film such as urethane in non-limiting embodiments. It can be either a woven, non-woven, or knit fabric. The softer more flexible and more conformable the substrate is, the more comfortable it is for the wearer of the mask. Stretch fabrics are optimal for wearer comfort, but other types of fabric may also be used.

The elastomer film barrier layer 22 is bonded to the fabric substrate layer 21 to provide a barrier for the silicone pressure sensitive adhesive (PSA) with active therapeutic ingredients layer 23. The elastomer film barrier layer 22 only allows migration of the active therapeutic ingredients toward the skin. The elastomer film barrier layer 22 can be made of any of several elastomeric polymers. In general, it should be very soft and thin so as not to create stiffness. In one or more embodiments, the elastomer film barrier layer 22 is a blend of polyurethane and polyester that is solvated and cast onto a release liner sheet, which can be used as the release liner 24. Extruded or calendared films may also be used in the construction disclosed herein. In a non-limiting embodiment, a formula for the elastomer film barrier layer 22 includes Lubrizol Pearlbond 360-28.5%, Bostik Vitel 3550-1.5%, and Cyclohexanone—70.0%.

The silicone pressure sensitive adhesive (PSA) with active therapeutic ingredients layer 23 includes a low trauma adhesive which is applied to the skin of the face, neck and/or décolletage area using light pressure on contact. The term “low trauma” relates to being non-painful or irritating while on the skin and while being applied to the skin and removed from the skin. The silicone PSA with active therapeutic ingredients layer 23 is composed of a pressure sensitive adhesive with more than one dispersed active therapeutic ingredient such as hyaluronic acid, CoQ10 (an antioxidant), Vitamin E and many other chemical, natural and homeopathic additives which gradually diffuse into the skin at any time of day or night while the mask is applied. The adhesive is silicone based. In one or more embodiments, the silicone PSA is a 100% solids addition cure type. It has the benefit of optimal biocompatibility with the skin (e.g. USP Class VI, and ISO 10993). Additionally, silicones do not support microbial growth due to their hydrophobicity. It has bio-durability, low surface tension, thermal stability, chemical stability, and good media resistance to a large number of solvents and chemicals. Silicones are sterilizable for single and repeated use using ethylene oxide. They have good weathering, ultra-violet (UV) ray and aging resistance. Being 100% solids creates a challenge to add the active therapeutic ingredients.

The adhesive that is applied to the elastomeric film barrier layer 22 is made with a 100% solids silicone polymer specifically “addition cure platinum catalyzed pressure sensitive silicone”. For example, the adhesive can be in a form of 100% solid, solvent-free silicone gel. The type of silicone PSA used in the adhesive is less damaging to the skin because it has a combination of low modulus and low peel strength but is still cohesive enough to remove cleanly. This type of cure chemistry silicone can be challenging due to its platinum catalyst. The platinum catalyst can be easily poisoned. Once poisoned, the silicone PSA will not cure. One specific inventive technology for the process and method of this application for incorporating the active therapeutic ingredients into the silicone for transdermal applications is disclosed herein. This technology provides for the release of the active therapeutic ingredients over the time the mask is worn, such as overnight. Active therapeutic ingredients that are liquids can be easily mixed into the silicone. Solid powdered ingredients require further processing steps to get homogeneously dispersed. These ingredients require milling to reduce particle size and to achieve dispersion. The silicone PSA serves as a vehicle for the transfer of the active therapeutic ingredients to the skin. In addition, the silicone serves as a barrier to prevent the evaporation of water from the skin overnight which slows down the dehydration of the skin and improves the condition of the skin.

One particular silicone used is an addition cure that includes a vinyl silicone, a MQ vinyl silicone, a silicone hydride, and a platinum catalyst. This formula is naturally, inherently tacky. The compounding process begins with a 100% solids two component silicone adhesive into which active therapeutic ingredients that are liquids and powders of high viscosities are weighed out and mixed into the vinyl silicone side. The mixture is further homogenized by milling. The preferred milling method is 3 roll milling. Media milling is also an option. Three roll milling using a milling machine with three physical rolls which turn at progressively faster speeds as the silicone mixture passes through the milling machine. Milling causes sheering of the particles within the composition meaning that the particles get broken down and dispersed more homogeneously into the silicone adhesive, which acts as a matrix. After the milling, the homogeneous compound is mixed with the silicone hydride and platinum ingredients. This mixture is reactive and has to be coated quickly after mixing (e.g., within 120 hours). The fully mixed compound is transfer coated from a release liner onto the omnidirectional stretch substrate. This coated laminate may be further processed into a final shaped form for application to a selected skin area via cutting technologies such as plotter or die cutting. The silicone laminate parts can be shaped in various ways. The invention can be shaped as a full-face mask, a partial face mask, a forehead mask, a nose area mask, an eye area mask, a neck area mask, an upper chest area mask, a hands mask, a finger mask, a shoulder mask, a knee mask, a leg mask, or other skin area mask in non-limiting embodiments where cosmetic and aesthetic treatments, such as moisturizing, may be desired.

In one or more embodiments, a formula for the active therapeutic ingredients is as follows:

| | |
|---|---|
| Wacker Silpuran 2122 | 94.77% |
| High Molecular weight Hyaluronic Acid | 2.16% |
| Low Molecular Weight Hyaluronic Acid | 2.16% |
| Collagen | 0.44% |
| EGCG (Epigallocatechin Gallate) | 0.22% |
| Argireline | 0.12% |
| Bakuchiol | 0.12% |
| Vitamin E | 0.02% |

Other embodiments of the active therapeutic ingredients may contain but not limited to Retinol, Peptides, CoQ10 (ubiquinol), Niacinamide, Phyto Ceramides, Plant Based Squalane, Sea Buckthorne Oil, Evening PrimRose, Apigenin, Polyglutamic Acid, White Shiitake Mushroom, medium-chain triglycerides (MCT), Menthol, Cannabidiol (CBD) and derivatives, Cannabigerol (CBG), Cannabinol (CBN), and others. Other active therapeutic ingredients may include skin lightening ingredients, moisturization enhancing ingredients, and other anti-aging ingredients.

In one or more embodiments, the release liner 24 is a 5 mil thick PET film coated on one side with a fluorosilicone release coating to provide easy release from the silicone PSA with active therapeutic ingredients layer 23. Several other liner options may also be used.

The release liner 24 is configured to cover the silicone PSA with active therapeutic ingredients layer 23 and protect it from being contaminated and from sticking to anything that comes in contact with it while not in use. This allows the mask 2 to be reused several times and still provide desired effects. The silicone PSA with active therapeutic ingredients layer 23 is removed from the release liner 24 just before applying the silicone PSA with active therapeutic ingredients layer 23 to the skin. The release liner 24 can be saved and the silicone PSA with active therapeutic ingredients layer 23 is reapplied to the release liner 24 after removal from the skin to keep the layer 23 clean and ready for a second or third application or even more applications until the silicone PSA with active therapeutic ingredients layer 23 is no longer tacky or the active therapeutic ingredients are used up, depleted, or exhausted. In one or more embodiments, the release liner 24 is the original casting liner on which the silicone PSA with active therapeutic ingredients layer 23 was cast and transfer coated during a coating operation.

Materials for the release liner 24 can be cut into shapes that will be designed to apply to the face in places that need treatment such as the forehead and crow's feet areas. The cut pieces can have rounded corners and relief cuts or cut-outs to facilitate easy application.

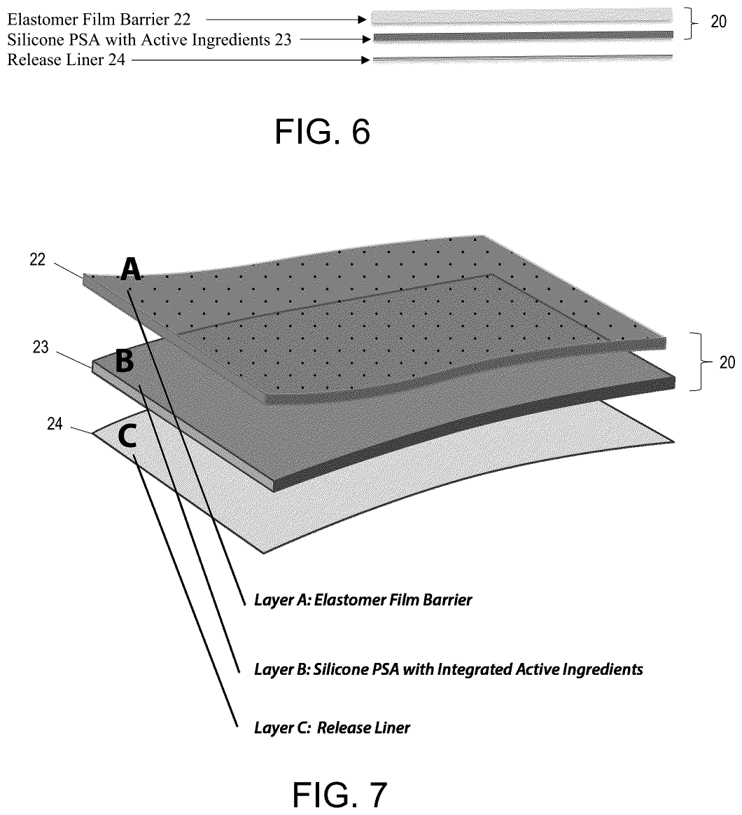

It can be appreciated that in an alternative embodiment the elastomeric film barrier layer 22 is made to be thicker (e.g., film thickness greater than 2 mil) and durable enough to be used without the need for the substrate layer 21 as illustrated in FIG. 6 in a cross-sectional view and in FIG. 7 in a perspective view. Hence, in this embodiment, the facial therapeutic mask 2 includes two layers—(1) the elastomer film barrier layer 22 and (2) the silicone PSA with active therapeutic ingredients layer 23.

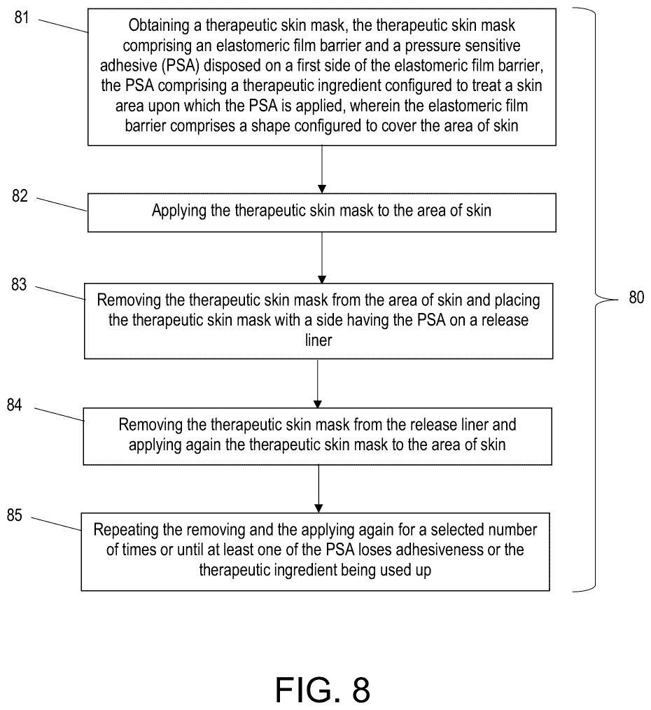

FIG. 8 is a flow chart for a method 80 for a therapeutic treatment of facial skin. Block 81 calls for obtaining a therapeutic skin mask, the therapeutic skin mask comprising an elastomeric film barrier layer and a pressure sensitive adhesive (PSA) disposed on a first side of the elastomeric film barrier layer, the PSA comprising a therapeutic ingredient configured to treat a skin area upon which the PSA is applied, wherein the elastomeric film barrier layer comprises a shape configured to cover the area of skin. Block 82 calls for applying the therapeutic skin mask to the area of skin. Block 83 calls for removing the therapeutic skin mask from the area of skin and placing the therapeutic skin mask with a side having the PSA on a release liner. Block 84 calls for removing the therapeutic skin mask from the release liner and applying again the therapeutic skin mask to the area of skin. Block 85 calls for repeating the removing and the applying again for a selected number of times or until at least one of the PSA loses adhesiveness or the therapeutic ingredient being deleted.

The method 80 may be implemented with the therapeutic skin mask having a substrate disposed on the elastomeric film barrier layer or with the therapeutic skin mask not having a substrate disposed on or coupled to the elastomeric film barrier layer.

Elements of the embodiments have been introduced with either the articles “a” or “an.” The articles are intended to mean that there are one or more of the elements. The terms “including” and “having” and the like are intended to be inclusive such that there may be additional elements other than the elements listed. The conjunction “or” when used with a list of at least two terms is intended to mean any term or combination of terms. The term “configured” relates to one or more structural limitations of a device that are required for the device to perform the function or operation for which the device is configured. The term “coupled” relates to being coupled directly or indirectly using an intermediate device. The terms “first” and “second” and like are used to distinguish terms and not to denote a particular order.

The flow diagram depicted herein is just an example. There may be many variations to this diagram or the steps (or operations) described therein without departing from the scope of the invention. For example, operations may be performed in another order or other operations may be performed at certain points without changing the specific disclosed sequence of operations with respect to each other. All of these variations are considered a part of the claimed invention.

The disclosure illustratively disclosed herein may be practiced in the absence of any element which is not specifically disclosed herein.

While the invention has been described with reference to an exemplary embodiment or embodiments, it will be understood by those skilled in the art that various changes may be made and equivalents may be substituted for elements thereof without departing from the scope of the invention. In addition, many modifications may be made to adapt a particular situation or material to the teachings of the invention without departing from the essential scope thereof. Therefore, it is intended that the invention not be limited to the particular embodiment disclosed as the best mode contemplated for carrying out this invention, but that the invention will include all embodiments falling within the scope of the claims. Also, in the drawings and the description, there have been disclosed exemplary embodiments of the invention and, although specific terms may have been employed, they are unless otherwise stated used in a generic and descriptive sense only and not for purposes of limitation, the scope of the invention therefore not being so limited.

What is claimed is:

1. A therapeutic skin mask comprising:
an elastomeric film barrier layer; and
a pressure sensitive adhesive (PSA) disposed on a first side of the elastomeric film barrier layer, the PSA comprising a therapeutic ingredient configured to treat an area of skin upon which the PSA is applied;
wherein the elastomeric film barrier layer comprises a shape configured to cover the area of skin.

2. The therapeutic skin mask according to claim 1, wherein the elastomeric film barrier layer comprises a blend of polyurethane and polyester.

3. The therapeutic skin mask according to claim 1, wherein the PSA comprises a 100% solid, solvent-free silicone gel.

4. The therapeutic skin mask according to claim 1, wherein the therapeutic ingredient comprises at least one of high molecular weight hyaluronic acid, low molecular weight hyaluronic acid, collagen, EGCG, CoQ10, argireline, bakuchiol, or vitamin E.

5. The therapeutic skin mask according to claim 1, wherein the elastomeric film barrier layer is not directly or indirectly coupled to a substrate on a second side of the elastomeric film barrier layer opposing the first side.

6. The therapeutic skin mask according to claim 1, further comprising a fabric substrate disposed on a second side on the elastomeric film barrier layer opposite the first side.

7. The therapeutic skin mask according to claim 6, wherein the fabric substrate is stretchable in multiple directions.

8. The therapeutic skin mask according to claim 1, further comprising a release liner configured to be applied to the PSA side of the therapeutic skin mask in response to the therapeutic skin mask not being used.

9. The therapeutic skin mask according to claim 8, wherein the release liner comprises a PET film coated on one side with a fluorosilicone release coating.

10. The therapeutic skin mask according to claim 1, wherein the shape of the elastomeric film barrier layer is such that the elastomeric film barrier layer is configured to cover at least one of a face, a partial area of the face, a neck, a hand, a knee, or a décolletage area.

11. The therapeutic skin mask according to claim 10, wherein the elastomeric film barrier layer is configured to cover the face and defines one or more openings for eyes, nostrils, or a mouth.

12. The therapeutic skin mask according to claim 10, wherein the shape comprises contours conforming to an area under an eye.

13. A method for a therapeutic treatment an area of skin, the method comprising:
obtaining a therapeutic skin mask, the therapeutic skin mask comprising an elastomeric film barrier layer and a pressure sensitive adhesive (PSA) disposed on a first side of the elastomeric film barrier layer, the PSA comprising a therapeutic ingredient configured to treat a skin area upon which the PSA is applied, wherein the elastomeric film barrier layer comprises a shape configured to cover the area of skin; and
applying the therapeutic skin mask to the area of skin.

14. The method according to claim 13, further comprising:
removing the therapeutic skin mask from the area of skin; and
placing the therapeutic skin mask with a side having the PSA on a release liner.

15. The method according to claim 14, further comprising removing the therapeutic skin mask from the release liner and applying again the therapeutic skin mask to the area of skin.

16. The method according to claim 15, further comprising repeating the removing the therapeutic skin mask from the area of skin, and the applying again the therapeutic skin mask to the area of skin, for a selected number of times or until at least one of the PSA loses adhesiveness or the therapeutic ingredient is depleted.

17. The method according to claim 13, wherein the elastomeric film barrier layer is not directly or indirectly coupled to a substrate on a second side of the elastomeric film barrier layer opposing the first side.

18. The method according to claim 13, wherein a fabric substrate disposed on a second side on the elastomeric film barrier layer opposite the first side.
